SEQUENCE LISTING (6 sequences)

SEQ ID NO: 1 - protein, 9 aa, Artificial Sequence
  Description of Artificial Sequence Synthetic peptide
SAFFLFCSE

SEQ ID NO: 2 - protein, 18 aa, Artificial Sequence
  Description of Artificial Sequence Synthetic peptide
  MOD_RES (15)..(15): Ala, Gly, Val or Phe
  MOD_RES (16)..(16): Ala, Gly, Val or Cys
  MOD_RES (17)..(17): Ala, Gly, Val or Ser
  MOD_RES (18)..(18): Ala, Gly, Val or Glu
  See specification as filed for detailed description of substitutions and preferred embodiments
DPNAPKRPPSAFFLXXXX

SEQ ID NO: 3 - RNA, 19 nt, Artificial Sequence
  Description of Artificial Sequence Synthetic oligonucleotide
uuauaaugacuggauguuc

SEQ ID NO: 4 - RNA, 19 nt, Artificial Sequence
  Description of Artificial Sequence Synthetic oligonucleotide
gucugguguaugaaggguu

SEQ ID NO: 5 - RNA, 19 nt, Artificial Sequence
  Description of Artificial Sequence Synthetic oligonucleotide
cuccuauuuugguuuaugc

SEQ ID NO: 6 - RNA, 19 nt, Artificial Sequence
  Description of Artificial Sequence Synthetic oligonucleotide
gcagcgucuuucagugcuu

(19) United States
(12) Patent Application Publication
(10) Pub. No.: US 2018/0325948 A1
(43) Pub. Date: Nov. 15, 2018

(54) TUMOR ANTIGEN MODIFIED DENDRITIC CELLS

(71) Applicants: Thomas ICHIM, San Diego, CA (US); JOSE JAVIER LOPEZ GONZALEZ, Chula Vista, CA (US); Santosh KESARI, Santa Monica, CA (US); Boris MINEV, San Diego, CA (US)

(72) Inventors: Thomas ICHIM, San Diego, CA (US); JOSE JAVIER LOPEZ GONZALEZ, Chula Vista, CA (US); Santosh KESARI, Santa Monica, CA (US); Boris MINEV, San Diego, CA (US)

(21) Appl. No.: 15/351,400

(22) Filed: Nov. 14, 2016

Related U.S. Application Data
(60) Provisional application No. 62/254,146, filed on Nov. 11, 2015.

(51) Int. Cl. A61K 35/15; A61K 39/00; A61P 35/00; A61K 38/03

(52) U.S. Cl. CPC A61K 35/15; A61K 39/001118; A61P 35/00; A61K 2039/572; A61K 39/001108; A61K 38/03; A61K 39/001109

(57) ABSTRACT

Treatment of cancer is disclosed through administration of proteins or specific peptides that are uptaken by dendritic cells, wherein said dendritic cells as subsequently activated and administered, in vivo, in a matter eliciting monocyte or dendritic cell migration in order to allow uptake of said administrated proteins or peptides, followed by administration of a maturation signal in vivo. Alternatively said immature dendritic cells are grown ex vivo in an autologous or semiallogenenic manner derived utilizing conventional means and pulsed with tumor peptides disclosed herein and subsequently administered. In some embodiments of the invention said dendritic cells are treated with epigenetic modifiers to enhance antigen presentation. The invention provides for treatment of cancer through induction of anticancer immunity and/or immunity towards tumor associated blood vessels.

### CROSS-REFERENCE TO RELATED APPLICATIONS

The present invention claims priority to provisional U.S. patent application 62/254,146, filed Nov. 11, 2015, which is hereby incorporated in its entirety including all tables, figures, and claims.

### FIELD OF THE INVENTION

The invention pertains to the field of cancer therapy, more specifically the invention pertains to the field of cancer immunotherapy. Specifically the invention pertains to the field of augmenting cancer immune responses through sequential administration of tumor antigens, in an environment suitable for antigen presenting cell uptake, followed by antigen presenting cell migration towards lymph nodes, following upregulation of antigen presenting cell activity through administration of an agent or plurality of agents capable of upregulating antigen presentation.

### BACKGROUND

The use of the immune system to treat cancer is theoretically appealing due to the possibility of low toxicity, immunological memory, and ability to attack metastatic disease. Early studies suggested that vaccination to tumor antigens and tumors themselves may be possible. Specifically, Prehn back in 1957 [1], obtained murine tumors and exposed them to irradiation to increase immunogenicity. When these tumors were implanted into animals they were rejected. Subsequent administration of the original tumors resulted in rejection of the tumors, thus suggesting that tumor specific antigens exist, which can stimulate immunity, especially subsequent to addition of a cellular stress such as irradiation. Twenty years later, using the same system it was demonstrated that cytotoxic T cells infiltrated the tumors that were implanted after rejection of the radiation induced tumors, thus demonstrating conclusively that rejection was immunologically mediated, despite the fact that the tumors were syngeneic [2]. In humans, one of the original observations of immunological response to neoplasia was in patients with paraneoplastic disease in which immune response to breast cancer antigens results in a multiple sclerosis-like disease caused by cross reactive immunity to neural antigens that are found on the breast cancer [3, 4]. Specific identification of tumor antigens on a molecular basis led to the discovery that some of the antigens are either self-proteins aberrantly expressed, or mutations of self proteins [5-8].

Originally observations were made in patients bearing metastatic melanomas, and then subsequently in other tumors, that the tumors are infiltrated with various immunological components. These tumor infiltrating lymphocytes (TILs), contain populations of cells and individual clones that demonstrate tumor specificity; they lyse autologous tumor cells but not natural killer targets, allogeneic tumor cells, or autologous fibroblasts [9-13].

By isolating and expanding TILs in vitro, and then molecularly identifying what they are responding to, a variety of the well-known tumor agents have been discovered such as MAGE-1 [13], and MAGE-3 [14], GAGE-1 [15], MART-1 [16], Melan-A [17], gp100 [18, 19], gp75 (TRP-2) [20, 21], tyrosinase [22], NY-ESO-1 [23], mutated p16 [24], and beta catenin [25]. It is interesting that in the case of some antigens, such as gp75, the peptide that elicits tumor rejection results from translation of an alternative open reading frame of the same gene. Thus, the gp75 gene encodes two completely different polypeptides, gp75 as an antigen recognized by immunoglobulin G antibodies in sera from a patient with cancer, and a 24-amino acid product as a tumor rejection antigen recognized by T cells [26]. Peptides used for immunization generally are 8-9 amino acids which have been demonstrated to be displayed in association with class I MHC molecules for recognition by T cells [27], and tumor cells have been shown to express these naturally processed epitopes.

Despite the intellectual appeal of peptide based cancer vaccines, the response rate has been disappointingly low. According to a review by Steven Rosenberg's group at the NIH, the rate of objective response out of 440 patients treated his institute was a dismal 2.6% [28].

The current invention provides means to increase efficacy of peptide vaccines.

### DESCRIPTION OF THE INVENTION

When practicing present invention it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To allow for the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

“antigen-presenting cells” or “APCs” are used to refer to autologous cells that express MHC Class I and/or Class II molecules that present antigens to T cells. Examples of antigen-presenting cells include, e.g., professional or non-professional antigen processing and presenting cells. Examples of professional APCs include, e.g., B cells, whole spleen cells, monocytes, macrophages, dendritic cells, fibroblasts or non-fractionated peripheral blood mononuclear cells (PMBC). Examples of hematopoietic APCs include dendritic cells, B cells and macrophages. Of course, it is understood that one of skill in the art will recognize that other antigen-presenting cells may be useful in the invention and that the invention is not limited to the exemplary cell types described herein. APCs may be “loaded” with an antigen that is pulsed, or loaded, with antigenic peptide or recombinant peptide derived from one or more antigens. In one embodiment, a peptide is the antigen and is generally antigenic fragment capable of inducing an immune response that is characterized by the activation of helper T cells, cytolytic T lymphocytes (cytolytic T cells or CTLs) that are directed against a malignancy or infection by a mammal. In one, embodiment the peptide includes one or more peptide fragments of an antigen that are presented by class I MHC or class II MHC molecules. The skilled artisan will recognize that peptides or protein fragments that are one or more fragments of other antigens may used with the present invention and that the invention is not limited to the exemplary peptides, tumor cells, cell clones, cell lines, cell supernatants, cell membranes, and/or antigens that are described herein.

“dendritic cell” or “DC” refer to all DCs useful in the present invention, that is, DC is various stages of differentiation, maturation and/or activation. In one embodiment of the present invention, the dendritic cells and responding T cells are derived from healthy volunteers. In another embodiment, the dendritic cells and T cells are derived from patients with cancer or other forms of tumor disease. In yet another embodiment, dendritic cells are used for either autologous or allogeneic application.

“effective amount” refers to a quantity of an antigen or epitope that is sufficient to induce or amplify an immune response against a tumor antigen, e.g., a tumor cell. “vaccine” refers to compositions that affect the course of the disease by causing an effect on cells of the adaptive immune response, namely, B cells and/or T cells. The effect of vaccines can include, for example, induction of cell mediated immunity or alteration of the response of the T cell to its antigen.

“immunologically effective” refers to an amount of antigen and antigen presenting cells loaded with one or more heat-shocked and/or killed tumor cells that elicit a change in the immune response to prevent or treat a cancer. The amount of antigen-loaded and/or antigen-loaded APCs inserted or reinserted into the patient will vary between individuals depending on many factors. For example, different doses may be required for an effective immune response in a human with a solid tumor or a metastatic tumor.

As used herein, the term “cancer cell” refers to a cell that exhibits an abnormal morphological or proliferative phenotype. The cancer cell may form part of a tumor, in which case it may be defined as a tumor cell. In vitro, cancer cells are characterized by anchorage independent cell growth, loss of contact inhibition and the like, as is known to the skilled artisan. As compared to normal cells, cancer cells may demonstrate abnormal new growth of tissue, e.g., a solid tumor or cells that invade surrounding tissue and metastasize to other body sites. A tumor or cancer “cell line” is generally used to describe those cells that are immortal and that may be grown in vitro. A primary cell is often used to describe a cell that is in primary culture, that is, it is freshly isolated from a patient, tissue or tumor. A cell clone will generally be used to describe a cell that has been isolated or cloned from a single cell and may or may not have been passed in in vitro culture. Examples of in vitro cancer cell lines useful for the practice of the invention as an antigen source include: J82, RT4, ScaBER, T24, TCCSUP, 5637 Carcinoma, SK-N-MC Neuroblastoma, SK-N-SH Neuroblastoma, SW 1088 Astrocytoma, SW 1783 Astrocytoma, U-87 MG Glioblastoma, astrocytoma, grade III, U-118 MG Glioblastoma, U-138 MG Glioblastoma, U-373 MG Glioblastoma, astrocytoma, grade III, Y79 Retinoblastoma, BT-20 Carcinoma, breast, BT-474 Ductal carcinoma, breast, MCF7 Breast adenocarcinoma, pleural effusion, MDA-MB-134-V Breast, ductal carcinoma, pleural I effusion, MDA-MD-157 Breast medulla, carcinoma, pleural effusion, MDA-MB-175-VII Breast, ductal carcinoma, pleural Effusion, MDA-MB-361 Adenocarcinoma, breast, metastasis to brain, SK-BR-3 Adenocarcinoma, breast, malignant pleural effusion, C-33 A Carcinoma, cervix, HT-3 Carcinoma, cervix, metastasis to lymph node

ME-180 Epidermoid carcinoma, cervix, metastasis to omentum, MEL-175 Melanoma, MEL-290 Melanoma, HLA-A*0201 Melanoma cells, MS751 Epidermoid carcinoma, cervix, metastasis to lymph

Node, SiHa Squamous carcinoma, cervix, JEG-3 Choriocarcinoma, Caco-2 Adenocarcinoma, colon

HT-29 Adenocarcinoma, colon, moderately well-differentiated grade II, SK-CO-1 Adenocarcinoma, colon, ascites, HuTu 80 Adenocarcinoma, duodenum, A-253 Epidermoid carcinoma, submaxillary gland

FaDu Squamous cell carcinoma, pharynx, A-498 Carcinoma, kidney, A-704 Adenocarcinoma, kidney

Caki-1 Clear cell carcinoma, consistent with renal primary, metastasis to skin, Caki-2 Clear cell carcinoma, consistent with renal primary, SK-NEP-1 Wilms' tumor, pleural effusion, SW 839 Adenocarcinoma, kidney, SK-HEP-1 Adenocarcinoma, liver, ascites, A-427 Carcinoma, lung

Calu-1 Epidermoid carcinoma grade III, lung, metastasis to pleura, Calu-3 Adenocarcinoma, lung, pleural effusion, Calu-6 Anaplastic carcinoma, probably lung, SK-LU-1 Adenocarcinoma, lung consistent with poorly differentiated, grade III, SK-MES-1 Squamous carcinoma, lung, pleural effusion, SW 900 Squamous cell carcinoma, lung, EB1 Burkitt lymphoma, upper maxilia, EB2 Burkitt lymphoma, ovary

P3HR-1 Burkitt lymphoma, ascites, HT-144 Malignant melanoma, metastasis to subcutaneous tissue

Malme-3M Malignant melanoma, metastasis to lung, RPMI-7951 Malignant melanoma, metastasis to lymph node, SK-MEL-1 Malignant melanoma, metastasis to lymphatic system, SK-MEL-2 Malignant melanoma, metastasis to skin of thigh, SK-MEL-3 Malignant melanoma, metastasis to lymph node

SK-MEL-5 Malignant melanoma, metastasis to axillary node, SK-MEL-24 Malignant melanoma, metastasis to node, SK-MEL-28 Malignant melanoma, SK-MEL-31 Malignant melanoma, Caov-3 Adenocarcinoma, ovary, consistent with primary, Caov-4 Adenocarcinoma, ovary, metastasis to subserosa of fallopian tube, SK-OV-3 Adenocarcinoma, ovary, malignant ascites, SW 626 Adenocarcinoma, ovary, Capan-1 Adenocarcinoma, pancreas, metastasis to liver, Capan-2 Adenocarcinoma, pancreas, DU 145 Carcinoma, prostate, metastasis to brain, A-204 Rhabdomyosarcoma, Saos-2 Osteogenic sarcoma, primary, SK-ES-1 Anaplastic osteosarcoma versus Swing sarcoma, SK-LNS-1 Leiomyosarcoma, vulva, primary, SW 684 Fibrosarcoma, SW 872 Liposarcoma

SW 982 Axilla synovial sarcoma, SW 1353 Chondrosarcoma, humerus, U-2 OS Osteogenic sarcoma, bone primary, Malme-3 Skin fibroblast, KATO III Gastric carcinoma, Cate-1B Embryonal carcinoma, testis, metastasis to lymph node, Tera-1 Embryonal carcinoma, Tera-2 Embryonal carcinoma, SW579 Thyroid carcinoma, AN3 CA Endometrial adenocarcinoma, metastatic, HEC-1-A Endometrial adenocarcinoma

HEC-1-B Endometrial adenocarcinoma, SK-UT-1 Uterine, mixed mesodermal tumor, consistent with

leiomyosarcomagrade III, SK-UT-1B Uterine, mixed mesodermal tumor, Sk-Me128 Melanoma

SW 954 Squamous cell carcinoma, vulva, SW 962 Carcinoma, vulva, lymph node metastasis, NCI-H69 Small cell carcinoma, lung, NCI-H128 Small cell carcinoma, lung, BT-483 Ductal carcinoma, breast

BT-549 Ductal carcinoma, breast, DU4475 Metastatic cutaneous nodule, breast carcinoma

HBL-100 Breast, Hs 578Bst Breast, Hs 578T Ductal carcinoma, breast, MDA-MB-330 Carcinoma, breast

MDA-MB-415 Adenocarcinoma, breast, MDA-MB-435s Ductal carcinoma, breast, MDA-MB-436 Adenocarcinoma, breast, MDA-MB-453 Carcinoma, breast, MDA-MB-468 Adenocarcinoma, breast

T-47D Ductal carcinoma, breast, pleural effusion, Hs 766T Carcinoma, pancreas, metastatic to lymph node, Hs 746T Carcinoma, stomach, metastatic to left leg, Hs 695T Amelanotic melanoma, metastatic to lymph node, Hs 683 Glioma, Hs 294T Melanoma, metastatic to lymph node, Hs 602 Lymphoma, cervical

JAR Choriocarcinoma, placenta, Hs 445 Lymphoid, Hodgkin's disease, Hs 700T Adenocarcinoma, metastatic to pelvis, H4 Neuroglioma, brain, Hs 696 Adenocarcinoma primary, unknown, metastatic

to bone-sacrum, Hs 913T Fibrosarcoma, metastatic to lung, Hs 729 Rhabdomyosarcoma, left leg, FHs 738Lu Lung, normal fetus, FHs 173We Whole embryo, normal, FHs 738B1 Bladder, normal fetus

NIH:OVCAR-3 Ovary, adenocarcinoma, Hs 67 Thymus, normal, RD-ES Ewing's sarcoma

ChaGo K-1 Bronchogenic carcinoma, subcutaneous, metastasis, human, WERI-Rb-1 Retinoblastoma

NCI-H446 Small cell carcinoma, lung, NCI-H209 Small cell carcinoma, lung, NCI-H146 Small cell carcinoma, lung, NCI-H441 Papillary adenocarcinoma, lung, NCI-H82 Small cell carcinoma, lung

H9 T-cell lymphoma, NCI-H460 Large cell carcinoma, lung, NCI-H596 Adenosquamous carcinoma, lung

NCI-H676B Adenocarcinoma, lung, NCI-H345 Small cell carcinoma, lung, NCI-H820 Papillary adenocarcinoma, lung, NCI-H520 Squamous cell carcinoma, lung, NCI-H661 Large cell carcinoma, lung

NCI-H510A Small cell carcinoma, extra-pulmonary origin, metastatic D283 Med Medulloblastoma

Daoy Medulloblastoma, D341 Med Medulloblastoma, AML-193 Acute monocyte leukemia

MV4-11 Leukemia biphenotype

“cancer cell antigen” refers to cells that have been stresses and killed in accordance with the present invention. Briefly, the cancer cells may be treated or stressed such that the cancer cell increases the expression of heat-shock proteins, such as HSP70, HSP60 and GP96, which are a class of proteins that are known to act as molecular chaperones for proteins that are or may be degraded. Generally, these heat-shock proteins will stabilize internal cancer cell antigens such that the cancer cells may include more highly immunogenic cancer cell-specific antigens.

[“contacted” and “exposed”, when applied to an antigen and APC, are used herein to describe the process by which an antigen is placed in direct juxtaposition with the APC. To achieve antigen presentation by the APC, the antigen is provided in an amount effective to “prime” the APCs to express antigen-loaded MHC class I and/or class II antigens on the cell surface.

“therapeutically effective amount” refers to the amount of antigen-loaded APCs that, when administered to an animal in combination, is effective to kill cancer cells within the animal. The methods and compositions of the present invention are equally suitable for killing a cancer cell or cells both in vitro and in vivo. When the cells to be killed are located within an animal, the present invention may be used in conjunction or as part of a course of treatment that may also include one or more anti-neoplastic agent, e.g., chemical, irradiation, X-rays, UV-irradiation, microwaves, electronic emissions, and the like. The skilled artisan will recognize that the present invention may be used in conjunction with therapeutically effective amount of pharmaceutical composition such a DNA damaging compound, such as, Adriamycin, 5-fluorouracil, etoposide, camptothecin, actinomycin-D, mitomycin C, cisplatin and the like. However, the present invention includes live cells that are going to activate other immune cells that may be affected by the DNA damaging agent. As such, any chemical and/or other course of treatment will generally be timed to maximize the adaptive immune response while at the same time aiding to kill as many cancer cells as possible.

“antigen-loaded dendritic cells,” “antigen-pulsed dendritic cells” and the like refer to DCs that have been contacted with an antigen, in this case, cancer cells that have been heat-shocked. Often, dendritic cells require a few hours, or up to a day, to process the antigen for presentation to naive and memory T-cells. It may be desirable to pulse the DC with antigen again after a day or two in order to enhance the uptake and processing of the antigen and/or provide one or more cytokines that will change the level of maturing of the DC. Once a DC has engulfed the antigen (e.g., pre-processed heat-shocked and/or killed cancer cells), it is termed an “antigen-primed DC”. Antigen-priming can be seen in DCs by immunostaining with, e.g., an antibody to the specific cancer cells used for pulsing. An antigen-loaded or pulsed DC population may be washed, concentrated, and infused directly into the patient as a type of vaccine or treatment against the pathogen or tumor cells from which the antigen originated. Generally, antigen-loaded DC are expected to interact with naive and/or memory T-lymphocytes in vivo, thus causing them to recognize and destroy cells displaying the antigen on their surfaces. In one embodiment, the antigen-loaded DC may even interact with T cells in vitro prior to reintroduction into a patient. The skilled artisan will know how to optimize the number of antigen-loaded DC per infusion, the number and the timing of infusions. For example, it will be common to infuse a patient with 1-2 million antigen-pulsed cells per infusion, but fewer cells may also induce the desired immune response.

The antigen-loaded DCs may be co-cultured with T-lymphocytes to produce antigen-specific T-cells. As used herein, the term “antigen-specific T-cells” refers to T-cells that proliferate upon exposure to the antigen-loaded APCs of the present invention, as well as to develop the ability to attack cells having the specific antigen on their surfaces. Such T-cells, e.g., cytotoxic T-cells, lyse target cells by a number of methods, e.g., releasing toxic enzymes such as granzymes and perforin onto the surface of the target cells or by effecting the entrance of these lytic enzymes into the target cell interior. Generally, cytotoxic T-cells express CD8 on their cell surface. T-cells that express the CD4 antigen CD4, commonly known as “helper” T-cells, can also help promote specific cytotoxic activity and may also be activated by the antigen-loaded APCs of the present invention. In certain embodiments, the cancer cells, the APCs and even the T-cells can be derived from the same donor whose MNC yielded the DC, which can be the patient or an HLA—or obtained from the individual patient that is going to be treated. Alternatively, the cancer cells, the APCs and/or the T-cells can be allogeneic.

The invention provides means of inducing an anti-cancer response in a mammal, comprising the steps of initially “priming” the mammal by administering an agent that causes local accumulation of antigen presenting cells. Subsequently, a tumor antigen is administered in the local area where said agents causing accumulation of antigen presenting cells is administered. A time period is allowed to pass to allow for said antigen presenting cells to traffic to the lymph nodes. Subsequently a maturation signal, or a plurality of maturation signals are administered to enhance the ability of said antigen presenting cell to activate adaptive immunity. In some embodiments of the invention activators of adaptive immunity are concurrently given, as well as inhibitors of the tumor derived inhibitors are administered to derepress the immune system.

In one embodiment priming of the patient is achieved by administration of GM-CSF subcutaneously in the area in which antigen is to be injected. Various scenarios are known in the art for administration of GM-CSF prior to administration, or concurrently with administration of antigen. The practitioner of the invention is referred to the following publications for dosage regimens of GM-CSF and also of peptide antigens [29-40]. Subsequent to priming, the invention calls for administration of tumor antigen. Various tumor antigens may be utilized, in one preferred embodiment, lysed tumor cells from the same patient area utilized. Means for generation of lyzed tumor cells are well known in the art and described in the following references [41-47]. One example method for generation of tumor lysate involves obtaining frozen autologous samples which are placed in hanks buffered saline solution (HBSS) and gentamycin 50 μg/ml followed by homogenization by a glass homogenizer. After repeated freezing and thawing, particle-containing samples are selected and frozen in aliquots after radiation with 25 kGy. Quality assessment for sterility and endotoxin content is performed before freezing. Cell lysates are subsequently administered into the patient in a preferred manner subcutaneously at the local areas where DC priming was initiated. After 12-72 hours, the patient is subsequently administered with an agent capable of inducing maturation of DC. Agents useful for the practice of the invention, in a preferred embodiment include BCG and HMGB1 peptide. Other useful agents include: a) histone DNA; b) imiqimod; c) beta-glucan; d) hsp65; e) hsp90; f) HMGB-1; g) lipopolysaccharide; h) Pam3CSK4; i) Poly I: Poly C; j) Flagellin; k) MALP-2; 1) Imidazoquinoline; m) Resiquimod; n) CpG oligonucleotides; o) zymosan; p) peptidoglycan; q) lipoteichoic acid; r) lipoprotein from gram-positive bacteria; s) lipoarabinomannan from mycobacteria; t) Polyadenylic-polyuridylic acid; u) monophosphoryl lipid A; v) single stranded RNA; w) double stranded RNA; x) 852A; y) rintatolimod; z) Gardiquimod; and aa) lipopolysaccharide peptides. The procedure is performed in a preferred embodiment with the administration of IDO silencing siRNA or shRNA containing the effector sequences a) UUAUAAUGACUGGAUGUUC (SEQ ID NO: 3); b) GUCUGGUGUAUGAAGGGUU (SEQ ID NO: 4); c) CUCCUAUUUUGGUUUAUGC (SEQ ID NO: 5) and d) GCAGCGUCUUUCAGUGCUU (SEQ ID NO: 6). siRNA or shRNA may be administered through various modalities including biodegradable matrices, pressure gradients or viral transfect. In another embodiment, autologous dendritic cells are generated and IDO is silenced, prior to, concurrent with or subsequent to silencing, said dendritic cells are pulsed with tumor antigen and administered systemically.

Culture of dendritic cells is well known in the art, for example, U.S. Pat. No. 6,936,468, issued to Robbins, et al., for the use of tolerogenic dendritic cells for enhancing tolerogenicity in a host and methods for making the same. Although the current invention aims to reduce tolerogenesis, the essential means of dendritic cell generation are disclosed in the patent. U.S. Pat. No. 6,734,014, issued to Hwu, et al., for methods and compositions for transforming dendritic cells and activating T cells. Briefly, recombinant dendritic cells are made by transforming a stem cell and differentiating the stem cell into a dendritic cell. The resulting dendritic cell is said to be an antigen presenting cell which activates T cells against MHC class I-antigen targets. Antigens for use in dendritic cell loading are taught in, e.g., U.S. Pat. No. 6,602,709, issued to Albert, et al. This patent teaches methods for use of apoptotic cells to deliver antigen to dendritic cells for induction or tolerization of T cells. The methods and compositions are said to be useful for delivering antigens to dendritic cells that are useful for inducing antigen-specific cytotoxic T lymphocytes and T helper cells. The disclosure includes assays for evaluating the activity of cytotoxic T lymphocytes. The antigens targeted to dendritic cells are apoptotic cells that may also be modified to express non-native antigens for presentation to the dendritic cells. The dendritic cells are said to be primed by the apoptotic cells (and fragments thereof) capable of processing and presenting the processed antigen and inducing cytotoxic T lymphocyte activity or may also be used in vaccine therapies. U.S. Pat. No. 6,455,299, issued to Steinman, et al., teaches methods of use for viral vectors to deliver antigen to dendritic cells. Methods and compositions are said to be useful for delivering antigens to dendritic cells, which are then useful for inducing T antigen specific cytotoxic T lymphocytes. The disclosure provides assays for evaluating the activity of cytotoxic T lymphocytes. Antigens are provided to dendritic cells using a viral vector such as influenza virus that may be modified to express non-native antigens for presentation to the dendritic cells. The dendritic cells are infected with the vector and are said to be capable of presenting the antigen and inducing cytotoxic T lymphocyte activity or may also be used as vaccines.

In one embodiment the invention teaches utilization of tumor cells with augmenting immunogenicity of said tumor cell is heat-shocking said cancer cell at a temperature of at least about 42.degree. C. for at least two hours to form heat shocked cancer cell. Furthermore, the invention teaches augmentation of immunogenicity by which one or more cancer cells are stressed by a method selected from the group consisting of heat shock, cold shock, glucose deprivation, oxygen deprivation, exposure to at least one drug that alter cell metabolism. Furthermore the invention provides the use of cell lines which are to be treated under conditions to augment immunogenicity, said cell lines selected from a group comprising of: J82, RT4, ScaBER, T24, TCCSUP, 5637 Carcinoma, SK-N-MC Neuroblastoma, SK-N-SH Neuroblastoma, SW 1088 Astrocytoma, SW 1783 Astrocytoma, U-87 MG Glioblastoma, astrocytoma, grade III, U-118 MG Glioblastoma, U-138 MG Glioblastoma, U-373 MG Glioblastoma, astrocytoma, grade III, Y79 Retinoblastoma, BT-20 Carcinoma, breast, BT-474 Ductal carcinoma, breast, MCF7 Breast adenocarcinoma, pleural effusion, MDA-MB-134-V Breast, ductal carcinoma, pleural I effusion, MDA-MD-157 Breast medulla, carcinoma, pleural effusion, MDA-MB-175-VII Breast, ductal carcinoma, pleural Effusion, MDA-MB-361 Adenocarcinoma, breast, metastasis to brain, SK-BR-3 Adenocarcinoma, breast, malignant pleural effusion, C-33 A Carcinoma, cervix, HT-3 Carcinoma, cervix, metastasis to lymph nodeME-180 Epidermoid carcinoma, cervix, metastasis to omentum, MEL-175 Melanoma, MEL-290 Melanoma, HLA-A*0201 Melanoma cells, MS751 Epidermoid carcinoma, cervix, metastasis to lymph

Node, SiHa Squamous carcinoma, cervix, JEG-3 Choriocarcinoma, Caco-2 Adenocarcinoma, colon

HT-29 Adenocarcinoma, colon, moderately well-differentiated grade II, SK-CO-1 Adenocarcinoma, colon, ascites, HuTu 80 Adenocarcinoma, duodenum, A-253 Epidermoid carcinoma, submaxillary gland

FaDu Squamous cell carcinoma, pharynx, A-498 Carcinoma, kidney, A-704 Adenocarcinoma, kidney

Caki-1 Clear cell carcinoma, consistent with renal primary, metastasis to skin, Caki-2 Clear cell carcinoma, consistent with renal primary, SK-NEP-1 Wilms' tumor, pleural effusion, SW 839 Adenocarcinoma, kidney, SK-HEP-1 Adenocarcinoma, liver, ascites, A-427 Carcinoma, lung

Calu-1 Epidermoid carcinoma grade III, lung, metastasis to pleura, Calu-3 Adenocarcinoma, lung, pleural effusion, Calu-6 Anaplastic carcinoma, probably lung, SK-LU-1 Adenocarcinoma, lung consistent with poorly differentiated, grade III, SK-MES-1 Squamous carcinoma, lung, pleural effusion, SW 900 Squamous cell carcinoma, lung, EB1 Burkitt lymphoma, upper maxilia, EB2 Burkitt lymphoma, ovary

P3HR-1 Burkitt lymphoma, ascites, HT-144 Malignant melanoma, metastasis to subcutaneous tissue

Malme-3M Malignant melanoma, metastasis to lung, RPMI-7951 Malignant melanoma, metastasis to lymph node, SK-MEL-1 Malignant melanoma, metastasis to lymphatic system, SK-MEL-2 Malignant melanoma, metastasis to skin of thigh, SK-MEL-3 Malignant melanoma, metastasis to lymph node

SK-MEL-5 Malignant melanoma, metastasis to axillary node, SK-MEL-24 Malignant melanoma, metastasis to node, SK-MEL-28 Malignant melanoma, SK-MEL-31 Malignant melanoma, Caov-3 Adenocarcinoma, ovary, consistent with primary, Caov-4 Adenocarcinoma, ovary, metastasis to subserosa of fallopian tube, SK-OV-3 Adenocarcinoma, ovary, malignant ascites, SW 626 Adenocarcinoma, ovary, Capan-1 Adenocarcinoma, pancreas, metastasis to liver, Capan-2 Adenocarcinoma, pancreas, DU 145 Carcinoma, prostate, metastasis to brain, A-204 Rhabdomyosarcoma, Saos-2 Osteogenic sarcoma, primary, SK-ES-1 Anaplastic osteosarcoma versus Swing sarcoma, SK-LNS-1 Leiomyosarcoma, vulva, primary, SW 684 Fibrosarcoma, SW 872 Liposarcoma

SW 982 Axilla synovial sarcoma, SW 1353 Chondrosarcoma, humerus, U-2 OS Osteogenic sarcoma, bone primary, Malme-3 Skin fibroblast, KATO III Gastric carcinoma, Cate-1B Embryonal carcinoma, testis, metastasis to lymph node, Tera-1 Embryonal carcinoma, Tera-2 Embryonal carcinoma, SW579 Thyroid carcinoma, AN3 CA Endometrial adenocarcinoma, metastatic, HEC-1-A Endometrial adenocarcinoma

HEC-1-B Endometrial adenocarcinoma, SK-UT-1 Uterine, mixed mesodermal tumor, consistent with

leiomyosarcomagrade III, SK-UT-1B Uterine, mixed mesodermal tumor, Sk-Me128 Melanoma

SW 954 Squamous cell carcinoma, vulva, SW 962 Carcinoma, vulva, lymph node metastasis, NCI-H69 Small cell carcinoma, lung, NCI-H128 Small cell carcinoma, lung, BT-483 Ductal carcinoma, breast

BT-549 Ductal carcinoma, breast, DU4475 Metastatic cutaneous nodule, breast carcinoma

HBL-100 Breast, Hs 578Bst Breast, Hs 578T Ductal carcinoma, breast, MDA-MB-330 Carcinoma, breast

MDA-MB-415 Adenocarcinoma, breast, MDA-MB-435s Ductal carcinoma, breast, MDA-MB-436 Adenocarcinoma, breast, MDA-MB-453 Carcinoma, breast, MDA-MB-468 Adenocarcinoma, breast

T-47D Ductal carcinoma, breast, pleural effusion, Hs 766T Carcinoma, pancreas, metastatic to lymph node, Hs 746T Carcinoma, stomach, metastatic to left leg, Hs 695T Amelanotic melanoma, metastatic to lymph node, Hs 683 Glioma, Hs 294T Melanoma, metastatic to lymph node, Hs 602 Lymphoma, cervical

JAR Choriocarcinoma, placenta, Hs 445 Lymphoid, Hodgkin's disease, Hs 700T Adenocarcinoma, metastatic to pelvis, H4 Neuroglioma, brain, Hs 696 Adenocarcinoma primary, unknown, metastatic

to bone-sacrum, Hs 913T Fibrosarcoma, metastatic to lung, Hs 729 Rhabdomyosarcoma, left leg, FHs 738Lu Lung, normal fetus, FHs 173We Whole embryo, normal, FHs 738B1 Bladder, normal fetus

NIH:OVCAR-3 Ovary, adenocarcinoma, Hs 67 Thymus, normal, RD-ES Ewing's sarcoma

ChaGo K-1 Bronchogenic carcinoma, subcutaneous, metastasis, human, WERI-Rb-1 Retinoblastoma

NCI-H446 Small cell carcinoma, lung, NCI-H209 Small cell carcinoma, lung, NCI-H146 Small cell carcinoma, lung, NCI-H441 Papillary adenocarcinoma, lung, NCI-H82 Small cell carcinoma, lung

H9 T-cell lymphoma, NCI-H460 Large cell carcinoma, lung, NCI-H596 Adenosquamous carcinoma, lung

NCI-H676B Adenocarcinoma, lung, NCI-H345 Small cell carcinoma, lung, NCI-H820 Papillary adenocarcinoma, lung, NCI-H520 Squamous cell carcinoma, lung, NCI-H661 Large cell carcinoma, lung

NCI-H510A Small cell carcinoma, extra-pulmonary origin, metastatic D283 Med Medulloblastoma

Daoy Medulloblastoma, D341 Med Medulloblastoma, AML-193 Acute monocyte leukemia

MV4-11 Leukemia biphenotype. The invention further teaches the augmentation of immunogenicity of a stressed cell, through the further inhibition of indolamine 2,3 deoxygenase (IDO) is performed locally during the immunization process. Said inhibition of IDO may be performed by administration of siRNA or shRNA. In one embodiment RNAi is induced by effector sequences are a combination of the nucleotides: a) UUAUAAUGACUGGAUGUUC (SEQ ID NO: 3); b) GUCUGGUGUAUGAAGGGUU (SEQ ID NO: 4); c) CUCCUAUUUUGGUUUAUGC (SEQ ID NO: 5) and d) GCAGCGUCUUUCAGUGCUU (SEQ ID NO: 6). In another embodiment immunogenicity is further increased in a cancer cell by treatment with a DNA methyltransferase inhibitor wherein said DNA methyltransferase inhibitors are selected from a group comprising of: 5-Azacytidine (which may be used at a concentration of 100 nM to 10 .mu.M), 5-Aza-2′-deoxycytidine (which may be used at a concentration of 100 nM to 10 .mu.M), 5-Fluoro-2′-deoxycytidine (which may be used at a concentration of 100 nM to 10 .mu.M), 5,6-Dihydro-5-azacytidine (which may be used at a concentration of 100 nM to 10 .mu.M), and Zebularine (which may be used at a concentration of 1 .mu.M to 10 mM). Exemplary non-nucleoside analogues include Hydralazine (which may be used at a concentration of 100 nM to 10 .mu.M), Procainamide (which may be used at a concentration of 1000 nM to 10 .mu.M), EGCG (which may be used at a concentration of 100 nM to 10 .mu.M), Psammaplin A (which may be used at a concentration of 100 nM to 10 .mu.M), MG98 (which may be used at a concentration of 100 nM to 10 .mu.M), and RG108 (which may be used at a concentration of 100 nM to 10 .mu.M). Further agents capable of epigenetically modifying tumors are used to treat tumor cells to augment immunogenicity. Said epigenetic modifying agents are histone deacetylases selected from a group comprising of: hydroxamic acids, Cyclic tetrapeptides and benzamides, and Benzamides. Exemplary short chain fatty acids include Butyrate (which may be used at a concentration of 1 .mu.M to 10 mM) and Valproic acid (which may be used at a concentration of 1 .mu.M to 10 mM). Exemplary hydroxamic acids include m-Carboxy cinnamic acid bishydroxamic acid (CBHA) (which may be used at a concentration of 100 nM to 10 .mu.M), Oxamflatin (which may be used at a concentration of 100 nM to 10 .mu.M), PDX 101 (which may be used at a concentration of 100 nM to 10 .mu.M), Pyroxamide (which may be used at a concentration of 1 nM to 10 .mu.M), Scriptaid (which may be used at a concentration of 100 nM to 10 .mu.M), Suberoylanilide hydroxamic acid (SAHA) (which may be used at a concentration of 100 nM to 10 .mu.M), Trichostatin A (TSA) (which may be used at a concentration of 1 nM to 10 .mu.M), LBH589 (which may be used at a concentration of 1 nM to 10 .mu.M), and NVP-LAQ824 (which may be used at a concentration of 1 nM to 10 .mu.M). Exemplary cyclic tetrapeptides and benzamides include Apicidin (which may be used at a concentration of 1 nM to 10 .mu.M), Depsipeptide (which may be used at a concentration of 100 nM to 10 .mu.M), TPX-HA analogue (CHAP) (which may be used at a concentration of 1 nM to 10 .mu.M), and Trapoxin (which may be used at a concentration of 1 nM to 10 .mu.M). Exemplary Benzamides include CI-994 (N-acetyldinaline) (which may be used at a concentration of 100 nM to 10 .mu.M) and MS-275 (which may be used at a concentration of 100 nM to 10 .mu.M).

### REFERENCES

1. Prehn, R. T. and J. M. Main, Immunity to methylcholanthrene-induced sarcomas. J Natl Cancer Inst, 1957. 18(6): p. 769-78.
2. Kripke, M. L., Antigenicity of murine skin tumors induced by ultraviolet light. J Natl Cancer Inst, 1974. 53(5): p. 1333-6.
3. Darnell, R. B., Onconeural antigens and the paraneoplastic neurologic disorders: at the intersection of cancer, immunity, and the brain. Proc Natl Acad Sci USA, 1996. 93(10): p. 4529-36.
4. Albert, M. L., et al., Tumor-specific killer cells in paraneoplastic cerebellar degeneration. Nat Med, 1998. 4(11): p. 1321-4.
5. Lurquin, C., et al., Structure of the gene of tum-transplantation antigen P91A: the mutated exon encodes a peptide recognized with Ld by cytolytic T cells. Cell, 1989. 58(2): p. 293-303.
6. Van den Eynde, B., et al., The gene coding for a major tumor rejection antigen of tumor P815 is identical to the normal gene of syngeneic DBA/2 mice. J Exp Med, 1991. 173(6): p. 1373-84.
7. Ghafouri-Fard, S. and M. H. Modarressi, Cancer-testis antigens: potential targets for cancer immunotherapy. Arch Iran Med, 2009. 12(4): p. 395-404.
8. Jager, D., E. Jager, and A. Knuth, Immune responses to tumour antigens: implications for antigen specific immunotherapy of cancer. J Clin Pathol, 2001. 54(9): p. 669-74.
9. Anichini, A., et al., Cytotoxic T lymphocyte clones from peripheral blood and from tumor site detect intratumor heterogeneity of melanoma cells. Analysis of specificity and mechanisms of interaction. J Immunol, 1989. 142(10): p. 3692-701.
10. Wolfel, T., et al., Lysis of human melanoma cells by autologous cytolytic T cell clones. Identification of human histocompatibility leukocyte antigen A2 as a restriction element for three different antigens. J Exp Med, 1989. 170(3): p. 797-810.
11. de Vries, J. E. and H. Spits, Cloned human cytotoxic T lymphocyte (CTL) lines reactive with autologous melanoma cells. I. In vitro generation, isolation, and analysis to phenotype and specificity. J Immunol, 1984. 132(1): p. 510-9.
12. Somasundaram, R., et al., CD8+, HLA-unrestricted, cytotoxic T-lymphocyte line against malignant melanoma. J Transl Med, 2005. 3: p. 41.
13. Topalian, S. L., D. Solomon, and S. A. Rosenberg, Tumor-specific cytolysis by lymphocytes infiltrating human melanomas. J Immunol, 1989. 142(10): p. 3714-25.
14. Gaugler, B., et al., Human gene MAGE-3 codes for an antigen recognized on a melanoma by autologous cytolytic T lymphocytes. J Exp Med, 1994. 179(3): p. 921-30.
15. Van den Eynde, B., et al., A new family of genes coding for an antigen recognized by autologous cytolytic T lymphocytes on a human melanoma. J Exp Med, 1995. 182(3): p. 689-98.
16. Kawakami, Y., et al., Cloning of the gene coding for a shared human melanoma antigen recognized by autologous T cells infiltrating into tumor. Proc Natl Acad Sci USA, 1994. 91(9): p. 3515-9.
17. Coulie, P. G., et al., A new gene coding for a differentiation antigen recognized by autologous cytolytic T lymphocytes on HLA-A2 melanomas. J Exp Med, 1994. 180(1): p. 35-42.
18. Bakker, A. B., et al., Melanocyte lineage-specific antigen gp100 is recognized by melanoma-derived tumor-infiltrating lymphocytes. J Exp Med, 1994. 179(3): p. 1005-9.
19. Kawakami, Y., et al., Identification of a human melanoma antigen recognized by tumor-infiltrating lymphocytes associated with in vivo tumor rejection. Proc Natl Acad Sci USA, 1994. 91(14): p. 6458-62.
20. Wang, R. F., et al., Identification of a gene encoding a melanoma tumor antigen recognized by HLA-A31-restricted tumor-infiltrating lymphocytes. J Exp Med, 1995. 181(2): p. 799-804.
21. Wang, R. F., et al., Identification of TRP-2 as a human tumor antigen recognized by cytotoxic T lymphocytes. J Exp Med, 1996. 184(6): p. 2207-16.
22. Brichard, V., et al., The tyrosinase gene codes for an antigen recognized by autologous cytolytic T lymphocytes on HLA-A2 melanomas. J Exp Med, 1993. 178(2): p. 489-95.
23. Jager, E., et al., Simultaneous humoral and cellular immune response against cancer-testis antigen NY-ESO-1: definition of human histocompatibility leukocyte antigen (HLA)-A2-binding peptide epitopes. J Exp Med, 1998. 187(2): p. 265-70.
24. Wolfel, T., et al., A p16INK4a-insensitive CDK4 mutant targeted by cytolytic T lymphocytes in a human melanoma. Science, 1995. 269(5228): p. 1281-4.
25. Robbins, P. F., et al., A mutated beta-catenin gene encodes a melanoma-specific antigen recognized by tumor infiltrating lymphocytes. J Exp Med, 1996. 183(3): p. 1185-92.
26. Wang, R. F., et al., Utilization of an alternative open reading frame of a normal gene in generating a novel human cancer antigen. J Exp Med, 1996. 183(3): p. 1131-40.
27. Townsend, A. R., F. M. Gotch, and J. Davey, Cytotoxic T cells recognize fragments of the influenza nucleoprotein. Cell, 1985. 42(2): p. 457-67.
28. Rosenberg, S. A., J. C. Yang, and N. P. Restifo, Cancer immunotherapy: moving beyond current vaccines. Nat Med, 2004. 10(9): p. 909-15.
29. Middleton, G., et al., Gemcitabine and capecitabine with or without telomerase peptide vaccine GV1001 in patients with locally advanced or metastatic pancreatic cancer (TeloVac): an open-label, randomised, phase 3 trial. Lancet Oncol, 2014. 15(8): p. 829-40.
30. Mittendorf, E. A., et al., Final report of the phase I/II clinical trial of the E75 (nelipepimut-S) vaccine with booster inoculations to prevent disease recurrence in high-risk breast cancer patients. Ann Oncol, 2014. 25(9): p. 1735-42.
31. Rahma, O. E., et al., The immunological and clinical effects of mutated ras peptide vaccine in combination with IL-2, GM-CSF, or both in patients with solid tumors. J Transl Med, 2014. 12: p. 55.
32. Clancy-Thompson, E., et al., Peptide vaccination in Montanide adjuvant induces and GM-CSF increases CXCR3 and cutaneous lymphocyte antigen expression by tumor antigen-specific CD8 T cells. Cancer Immunol Res, 2013. 1(5): p. 332-9.
33. Sonpavde, G., et al., HLA-restricted NY-ESO-1 peptide immunotherapy for metastatic castration resistant prostate cancer. Invest New Drugs, 2014. 32(2): p. 235-42.
34. Geynisman, D. M., et al., A randomized pilot phase I study of modified carcinoembryonic antigen (CEA) peptide (CAP1-6D)/montanide/GM-CSF-vaccine in patients with pancreatic adenocarcinoma. J Immunother Cancer, 2013. 1: p. 8.
35. Tarhini, A. A., et al., Differing patterns of circulating regulatory T cells and myeloid-derived suppressor cells in metastatic melanoma patients receiving anti-CTLA4 antibody and interferon-alpha or TLR-9 agonist and GM-CSF with peptide vaccination. J Immunother, 2012. 35(9): p. 702-10.
36. Walter, S., et al., Multipeptide immune response to cancer vaccine IMA901 after single-dose cyclophosphamide associates with longer patient survival. Nat Med, 2012. 18(8): p. 1254-61.
37. Ohno, S., et al., Phase I trial of Wilms' Tumor 1 (WT1) peptide vaccine with GM-CSF or CpG in patients with solid malignancy. Anticancer Res, 2012. 32(6): p. 2263-9.
38. Tarhini, A. A., et al., Safety and immunogenicity of vaccination with MART-1 (26-35, 27L), gp100 (209-217, 210M), and tyrosinase (368-376, 370D) in adjuvant with PF-3512676 and GM-CSF in metastatic melanoma. J Immunother, 2012. 35(4): p. 359-66.
39. Schaefer, C., et al., Function but not phenotype of melanoma peptide-specific CD8(+) T cells correlate with survival in a multiepitope peptide vaccine trial (ECOG 1696). Int J Cancer, 2012. 131(4): p. 874-84.
40. Block, M. S., et al., Pilot study of granulocyte-macrophage colony-stimulating factor and interleukin-2 as immune adjuvants for a melanoma peptide vaccine. Melanoma Res, 2011. 21(5): p. 438-45.
41. Bapsy, P. P., et al., Open-label, multi-center, non-randomized, single-arm study to evaluate the safety and efficacy of dendritic cell immunotherapy in patients with refractory solid malignancies, on supportive care. Cytotherapy, 2014. 16(2): p. 234-44.
42. Reyes, D., et al., Tumour cell lysate-loaded dendritic cell vaccine induces biochemical and memory immune response in castration-resistant prostate cancer patients. Br J Cancer, 2013. 109(6): p. 1488-97.
43. Kamigaki, T., et al., Immunotherapy of autologous tumor lysate-loaded dendritic cell vaccines by a closed-flow electroporation system for solid tumors. Anticancer Res, 2013. 33(7): p. 2971-6.
44. Florcken, A., et al., Allogeneic partially HLA-matched dendritic cells pulsed with autologous tumor cell lysate as a vaccine in metastatic renal cell cancer: a clinical phase I/II study. Hum Vaccin Immunother, 2013. 9(6): p. 1217-27.
45. Cho, D. Y., et al., Adjuvant immunotherapy with whole-cell lysate dendritic cells vaccine for glioblastoma multiforme: a phase II clinical trial. World Neurosurg, 2012. 77(5-6): p. 736-44.
46. Alfaro, C., et al., Pilot clinical trial of type 1 dendritic cells loaded with autologous tumor lysates combined with GM-CSF, pegylated IFN, and cyclophosphamide for metastatic cancer patients. J Immunol, 2011. 187(11): p. 6130-42.
47. Fadul, C. E., et al., Immune response in patients with newly diagnosed glioblastoma multiforme treated with intranodal autologous tumor lysate-dendritic cell vaccination after radiation chemotherapy. J Immunother, 2011. 34(4): p. 382-9.

SEQUENCE LISTING:

SAFFLFCSE (SEQ ID NO: 1)
DPNAPKRPPSAFFLX.sub.1X.sub.2X.sub.3X.sub.4
(SEQ ID NO: 2)
UUAUAAUGACUGGAUGUUC (SEQ ID NO: 3)
GUCUGGUGUAUGAAGGGUU (SEQ ID NO: 4)
CUCCUAUUUUGGUUUAUGC (SEQ ID NO: 5)
GCAGCGUCUUUCAGUGCUU (SEQ ID NO: 6)

1. A method of generating a dendritic capable of stimulating T cells to kill cancer comprising the steps of; a) treating said dendritic cell with an epigenetic modifying agent; b) administering to said dendritic cell a tumor or tumor endothelial associated antigen; c) administering a chemoattracting agent to a patient in need of treatment; d) administering said dendritic cell to said patient after treatment of said dendritic cell in a manner to stimulate ability of said dendritic cell to stimulate a T cell response,

2. The method of claim 1, wherein said tumor antigen or tumor endothelial antigen is selected from a group comprising of: a) a peptide; b) an altered peptide ligand; c) a protein; d) a modified protein; e) a cell penetrating peptide conjugated to a peptide or protein; f) an mRNA encoding a peptide or protein; g) a plasmid encoding a peptide or protein; h) a viral vector encoding a peptide or protein; i) a tumor cell; j) an endothelial cell; k) a tumor cell treated with an agent capable of augmenting immunogenicity of said tumor cell; l) an endothelial cell treated with an agent capable of augmenting immunogenicity of said endothelial cell; m) an endothelial cell treated in a manner to induce expression of genes associated with tumor endothelial cells; and n) a tumor endothelial cell.

3. The method of claim 1, wherein said tumor antigen is selected from a group comprising of: a) Fos-related antigen 1; b) LCK; c) FAP; d) VEGFR2; e) NA17; f) PDGFR-beta; g) PAP; h) MAD-CT-2; i) Tie-2; j) PSA; k) protamine 2; l) legumain; m) endosialin; n) prostate stem cell antigen; o)carbonic anhydrase IX; p) STn; q) Page4; r) proteinase 3; s) GM3 ganglioside; t) tyrosinase; u) MART1; v) gp100; w) SART3; x) RGS5; y) SSX2; z) Globoll; aa) Tn; ab) CEA; ac) hCG; ad) PRAME; ae) XAGE-1; af) AKAP-4; ag) TRP-2; ah) B7H3; ai) sperm fibrous sheath protein; aj) CYP1B1; ak) HMWMAA; al) sLe(a); am) MAGE A1; an) GD2; ao) PSMA; ap) mesothelin; aq) fucosyl GM1; ar) GD3; as) sperm protein 17; at) NY-ESO-1; au) PAX5; av) AFP; aw) polysialic acid; ax) EpCAM; ay) MAGE-A3; az) mutant p53; ba) ras; bb) mutant ras; bc) NY-BR1; bd) PAX3; be) HER2/neu; bf) OY-TES1; bg) HPV E6 E7; bh) PLAC1; bi) hTERT; bj) BORIS; bk) ML-IAP; bl) idiotype of b cell lymphoma or multiple myeloma; bm) EphA2; bn) EGFRvIII; bo) cyclin B1; bp) RhoC; bq) androgen receptor; br) surviving; bs) MYCN; bt) wildtype p53; bu) LMP2; by) ETV6-AML; bw) MUC1; bx) BCR-ABL; by) ALK; bz) WT1; ca) ERG (TMPRSS2 ETS fusion gene); cb) sarcoma translocation breakpoint; cc) STEAP; cd) OFA/iLRP; and ce) Chondroitin sulfate proteoglycan 4 (CSPG4)

4. The method of claim 1, wherein said chemoattractant agent is an agent capable of increasing numbers of dendritic cells either through chemotaxis or through induction of monocyte differentiation into dendritic cells.

5. The method of claim 4, wherein said chemoattractant agent is GM-CSF.

6. The method of claim 4, wherein said chemoattractant agent is lymphotactin.

7. The method of claim 5, wherein GM-CSF is administered subcutaneously at a concentration of approximately between 7 micrograms to 700 micrograms.

8. The method of claim 5, wherein GM-CSF is administered subcutaneously at a concentration of approximately 70 micrograms.

9. The method of claim 5, wherein GM-CSF is administered approximately at two days before antigen immunization and on the day of antigen immunization at the same location as the peptide immunization will occur.

10. The method of claim 1, wherein said antigen immunization is performed at days 0, 30, 45, 60, 75, and 90.

11. The method of claim 1, wherein an adjuvant is administered together with said antigen.

12. The method of claim 11, wherein said adjuvant is selected from a group of adjuvants comprising of: a) a TLR agonist; b) Montanide; c) complete Freund's adjuvant; and d) incomplete Freund's adjuvant.

13. The method of claim 1, wherein a helper peptide or protein is coadministered with said antigen.

14. The method of claim 13, wherein said helper peptide is a PADRE peptide.

15. The method of claim 13, wherein said helper protein is KLH.

16. The method of claim 1, wherein said time period allowed for DC to traffic to lymph node is approximately between 1 hour to 200 hours.

17. The method of claim 1, wherein said time period allowed for DC to traffic to lymph node is approximately 48 hours.

18. The method of claim 1, wherein said maturation signal is selected from a group of compounds comprising of: a) HMGB1 peptide; b) a TLR agonist; c) interferon alpha; d) interferon gamma; and e) IL-18.

19. The method of claim 18, wherein said HMGB1 peptide is comprised of the amino acids SAFFLFCSE (SEQ ID NO: 1).

20. The method of claim 18, wherein said HMGB1 peptide is comprised of DPNAPKRPPSAFFLX.sub.1X.sub.2X.sub.3X.sub.4 (SEQ ID NO: 2) or a derivative thereof, Wherein when X1 is alanine (A), glycine (G), or valine (V) then X2 is C, X3 is S and X4 is E; Wherein when X2 is alanine (A), glycine (G), or valine (V) then X1 is F, X3 is S and X4 is E; Wherein when X3 is alanine (A), glycine (G), or valine (V) then X1 is F, X2 is C and X4 is E; or Wherein when X4 is alanine (A), glycine (G), or valine (V) then X1 is F, X2 is C and X3 is S.
